(19) United States
(12) Patent Application Publication
(10) Pub. No.: US 2024/0095323 A1
(43) Pub. Date: Mar. 21, 2024

(54) DISPLAY LAYOUT AND INTERACTIVE OBJECTS FOR PATIENT MONITORING

(71) Applicant: Masimo Corporation, Irvine, CA (US)

(72) Inventors: Massi Joe E. Kiani, Laguna Niguel, CA (US); Bilal Muhsin, Irvine, CA (US); Ammar Al-Ali, San Juan Capistrano, CA (US); Nicholas Evan Barker, Laguna Beach, CA (US); Chad A. DeJong, Los Angeles, CA (US); Omar Ahmed, Lake Forest, CA (US); Keith Ward Indorf, Lake Elsinore, CA (US); Steve Coon, Irvine, CA (US)

(21) Appl. No.: 18/474,071

(22) Filed: Sep. 25, 2023

Related U.S. Application Data
(63) Continuation of application No. 17/072,963, filed on Oct. 16, 2020, now Pat. No. 11,803,623.
(60) Provisional application No. 62/923,248, filed on Oct. 18, 2019.
(60) Provisional application No. 63/017,151, filed on Apr. 29, 2020.

(51) Int. Cl. G06F 21/32; G16H 10/60; G16H 40/20; G16H 40/67; G16H 50/50; G16H 80/00

(52) U.S. Cl. CPC G06F 21/32; G16H 10/60; G16H 40/20; G16H 40/67; G16H 50/50; G16H 80/00; A61B 5/0024

(57) ABSTRACT

A physiological patient monitoring system with a patient-facing interface is disclosed. The patient interface can be used by the patient to communicate with hospital staff without actually requesting attendance and can request attendance for specific purposes. The patient interface may also track patient treatment and inform patients of the details of their treatments.

### CROSS REFERENCE TO PRIORITY APPLICATIONS

Any and all applications for which a foreign or domestic priority claim is identified in the Application Data Sheet as filed with the present application are hereby incorporated by reference under 37 CFR § 1.57. This application is a continuation of U.S. patent application Ser. No. 17/072,963, filed Oct. 16, 2020, entitled “DISPLAY LAYOUT AND INTERACTIVE OBJECTS FOR PATIENT MONITORING ,” which claims the benefit of priority to U.S. Provisional Patent Application No. 62/923,248, filed Oct. 18, 2019, entitled “DISPLAY LAYOUT AND INTERACTIVE OBJECTS FOR PATIENT MONITORING,” and to U.S. Provisional Patent Application No. 63/017,151, filed Apr. 29, 2020, entitled “DISPLAY LAYOUT AND INTERACTIVE OBJECTS FOR PATIENT MONITORING.” All the foregoing are hereby incorporated by reference in their entireties herein.

### BACKGROUND

The present disclosure relates to display layouts and interactive objects for a physiological patient monitoring system.

Often patients have lackluster experiences at hospitals, regardless of the quality of treatment or the reputation of the hospital. Patients often do not feel informed enough about their treatments or do not feel they are receiving the right amount of attention. This leads to patients feeling confused or detached from their treatment. The problem is caused at least in part by the fact that the current hospital system has many inefficiencies that use care team members' time ineffectively. For example, most hospitals currently only have a general attendance button used for both non-emergencies, such as a request for a glass of water, as well as life-threatening events. Medical staff are thus wasting their time running between rooms for attendance requests that should be directed toward support staff. Further, entire teams of healthcare professionals take care of a single patient, but in the current hospital system, information is not shared in a way that allows every member to stay up to date on the patient's treatment and progress. Such gaps in knowledge mean not only that patients are not receiving answers when they ask questions, but there is a heightened risk of clinician error each time a new care team member arrives. Thus, there is a need for more effective and efficient channels of communication between hospital staff and patients.

### SUMMARY

For purposes of summarizing the disclosure, certain aspects, advantages and novel features are discussed herein. It is to be understood that not necessarily all such aspects, advantages, or features will be embodied in any particular configuration of the invention and an artisan would recognize from the disclosure herein a myriad of combinations of such aspects, advantages or features.

The disclosure describes a physiological patient monitoring system with a patient-facing interface. The patient interface can be used by the patient to communicate with hospital staff without actually requesting attendance and can request attendance for specific purposes such that physicians and nurses are not running room to room for non-medical emergency needs. The patient interface can also track patient treatment and informs patients of the details of their treatments, thereby increasing engagement.

### BRIEF DESCRIPTION OF THE DRAWINGS

Throughout the drawings, reference numbers may be re-used to indicate correspondence between referenced elements. The drawings are provided to illustrate example configurations described herein and are not intended to limit the scope of the disclosure.

FIG. 1 depicts a hospital patient using a portable device with a patient interface for a physiological monitoring system.

FIG. 2 is an illustrative interface for a default screen for a patient interface on portable devices in hospital rooms, according to some configurations.

FIG. 3 is an illustrative interface for a room control panel summon screen where the room control panel appears in full view while the remaining screen features are dimmed, according to some configurations.

FIG. 4 is an illustrative interface for a full screen view of a room control panel according to some configurations.

FIG. 5 is an illustrative interface displaying personalized information regarding a hospital building and a patient's care staff, according to some configurations.

FIG. 6 is an illustrative interface of different methods patients may utilize to communicate with hospital staff, according to some configurations.

FIGS. 7A-7C are illustrative interfaces showing how patients could communicate their levels of pain while requesting attendance, according to some configurations.

FIGS. 8A-8B are illustrative interfaces for a feature which allows patients to communicate with their assigned nurse via text messaging, according to some configurations.

FIG. 9 is an illustrative interface for a feature which allows patients to communicate with their assigned nurse via either audio or video communication, according to some configurations.

FIG. 10 is an illustrative interface for a feature which allows patients to record questions to ask hospital staff, according to some configurations.

FIGS. 11A-11B are illustrative interfaces showing medication prescription and administration details for a patient, according to some configurations.

FIGS. 12A-12B are illustrative interfaces showing treatment details and patient vitals, according to some configurations.

FIG. 13 is an illustrative interface showing a patient's daily goals, according to some configurations.

FIGS. 14A-14C are illustrative interfaces for a patient meal ordering feature, according to some configurations.

### DETAILED DESCRIPTION OF THE PREFERRED CONFIGURATION

The current hospital system has several inefficiencies, many of which are caused by ineffective communication. Such ineffective communication leads to negative patient experience because patients feel confused and detached from their treatment. Patients are ill-informed about the details of their treatment, and they cannot communicate their needs until a hospital staff member, who may not be the best person for the patient's particular need, is already in the room. The presently disclosed patient interface of a physiological patient monitoring system addresses the issue of inefficient communication by creating a portal through which patients can both track details about their treatment and communicate their exact needs to the most relevant members of the hospital staff. By fostering quick yet precise communication, the present system aims to improve patient hospital experiences and increase patient understanding and engagement in treatments. Furthermore, physical switches may be heard to reach or the wires may be cumbersome. Accordingly, the systems described herein can improve communication without requiring physical switches that a patient may need to try to access to get help. In some instances, there are no physical switches and/or wires to call a nurse or indicate status in the hospital room.

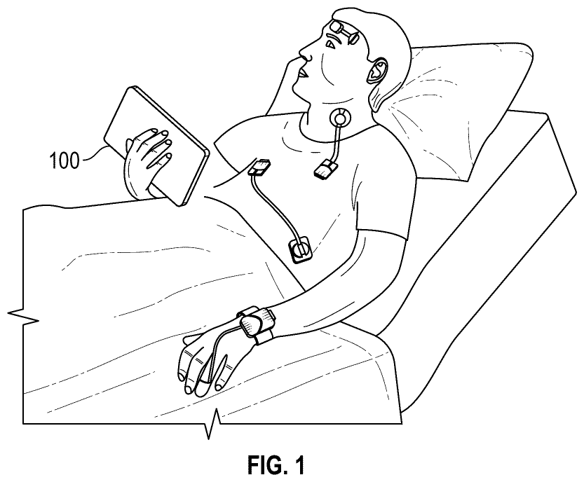

FIG. 1 depicts a hospital patient using a portable device 100 with a patient interface for a physiological patient monitoring system. The physiological patient monitoring system may be connected to patient monitoring equipment and a hospital data network such that the patient interface may be updated with live measurements, diagnoses, and treatment plans. Updates may occur continuously, automatically, and/or only when new data is manually entered. Patients may thus be more informed about and involved with their treatment and progress. Patients may also communicate with hospital staff and request attendance from their hospital beds through the physiological patient monitoring system. The system may route patient requests to the appropriate hospital department based on the content of the patient's request.

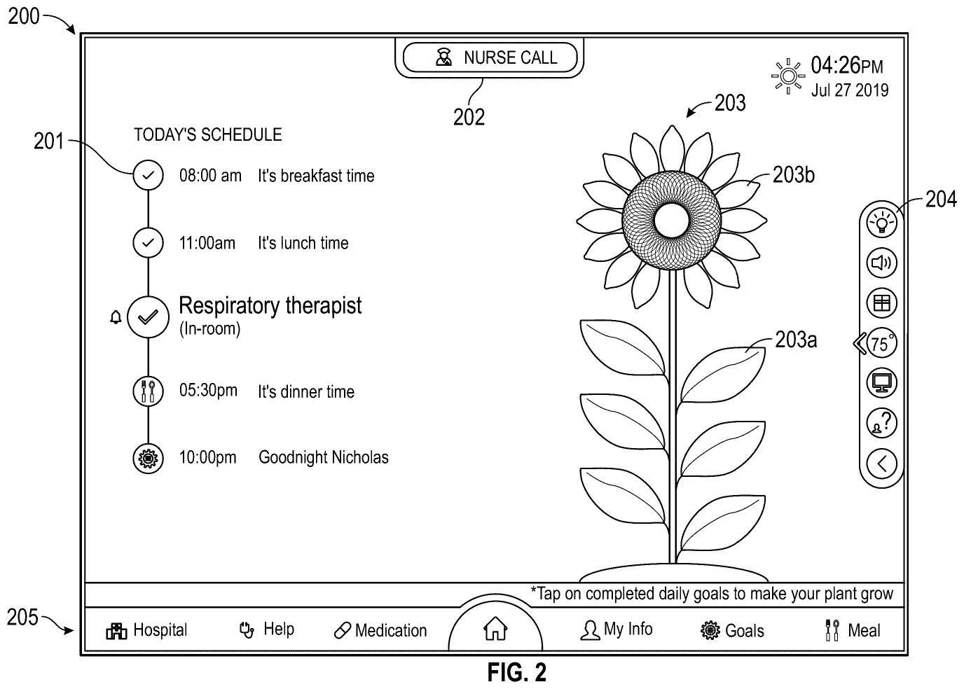

FIG. 2 illustrates an example default screen 200 for a patient interface for portable devices in hospital rooms. The default screen 200 may display a patient's daily schedule 201, nurse call button 202, progress measurement feature 203, room control panel 204, and application navigation panel 205. The application navigation panel 205 may be used to access all other pages of the application. To return to the default screen 200, the patient may simply tap on the home button in the navigation panel 205. For ease of access, the nurse call button 202 may remain in the same location on each screen of the application. The daily schedule 201 may show the time at which each task is set to occur. The daily schedule 201 may update automatically once the scheduled time for an event has passed, or it may only mark a task as complete when the patient, a healthcare provider, or both manually mark the task as complete. In some configurations, the progress measurement feature 203 may be shaped like a flower that grows as daily tasks are completed. A leaf 203a may be added for completed tasks and the flower 203b may bloom when all tasks are complete. Other configurations may assign different parts of the flower to completed tasks, contain more or less detailed parts of the flower, or may use a different graphical representation of patient progress altogether. Together, the daily schedule 201 and the progress measurement feature 203 can inform and motivate patients throughout their recovery. The room control panel 204 may be partially hidden when not in use.

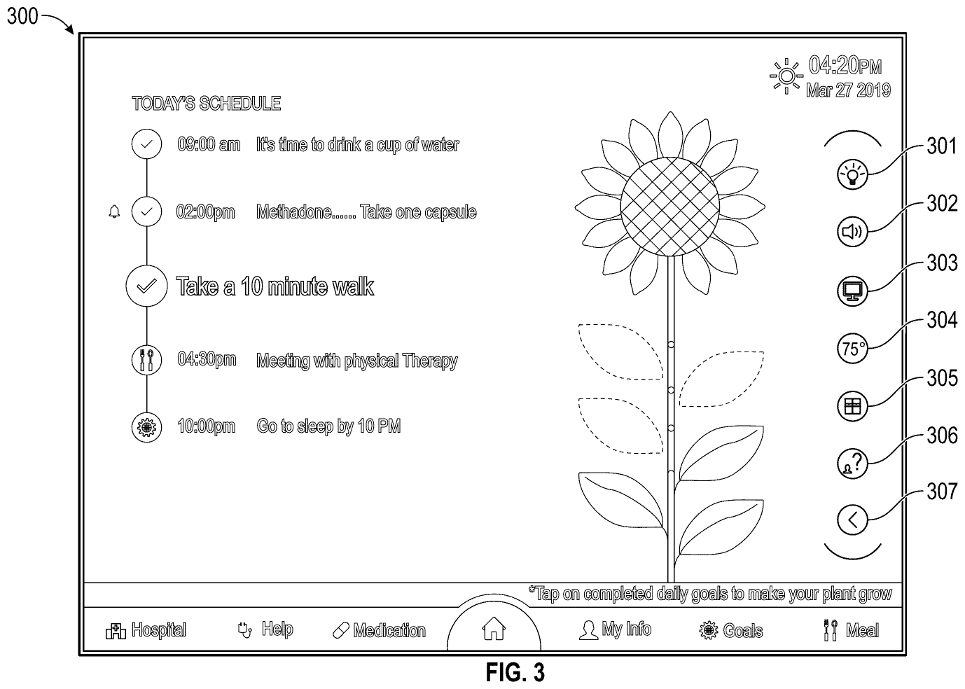

FIG. 3 shows an example room control panel summon screen 300 where the room control panel 204 shown in FIG. 2 may appear in full view while the remaining screen features are dimmed. The room control panel can allow patients to control the light 301, television or speaker volume 302, television 303, temperature 304, and window blinds 305 without calling hospital staff or even leaving their beds. The control panel may also include a help button 306 which explains how to use the application. The call-out button 307 can open the control panel as its own full screen.

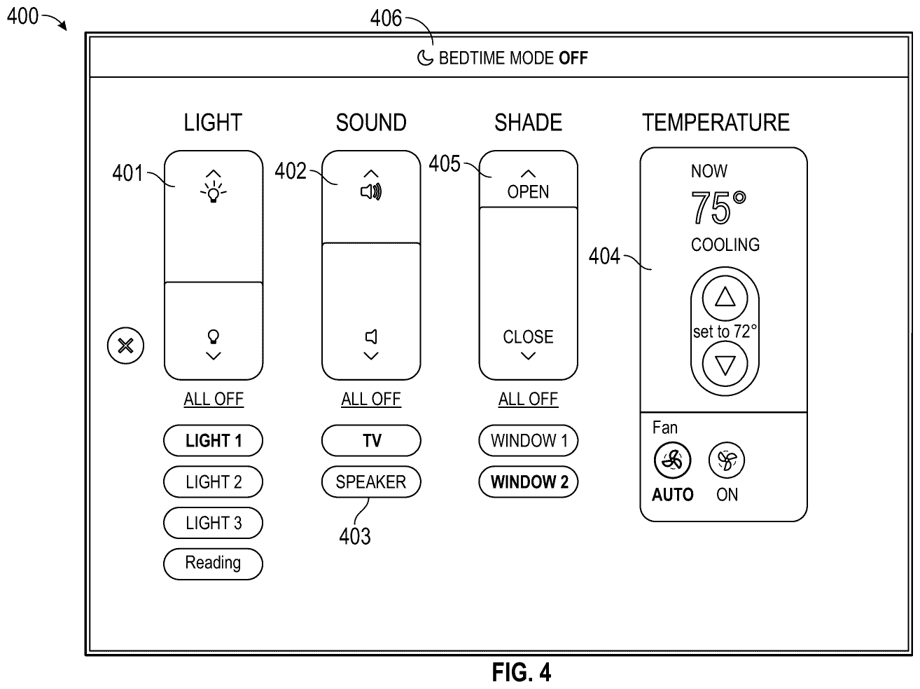

FIG. 4 shows an illustrative full screen view 400 of the room control panel 204 from FIG. 2. The same features that were in the sidebar view may appear on this screen in full: light 401, volume 402, television 403, temperature 404, and window blinds 405. The full screen view 400 may include detailed buttons that allow more specific control of the features, such as which individual light to adjust, which window's blind to move, or other such changes to individual ambient items. The full screen view 400 of the room control panel can have an extra bedtime mode button 406. Bedtime mode may darken the screen such that patients can sleep without blue light disruption. With greater control over their hospital room environment, patients may not need to call for hospital staff assistance as often, thereby freeing up healthcare providers to attend to more urgent matters. Patients may also feel more comfortable as they can make adjustments to their liking at any time.

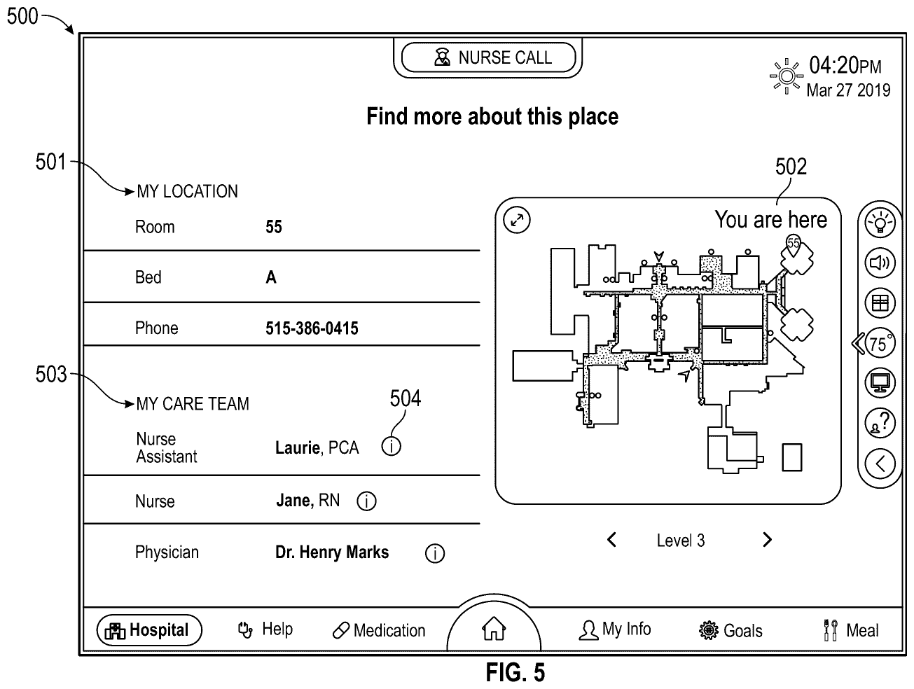

FIG. 5 illustrates an illustrative Hospital tab screen 500 which may display personalized information regarding the hospital building and the patient's care staff. The Hospital screen 500 may be accessed via the application navigation panel 205. The Hospital tab may show the patient's room information 501 and a hospital map 502. The patient's care team information 503 may also be included. The information button 504, when pressed, may create a pop-up or separate sub-screen that shares the individual team member's credentials, background, and any other information the team member chooses to share so that patients feel more connection with their care team.

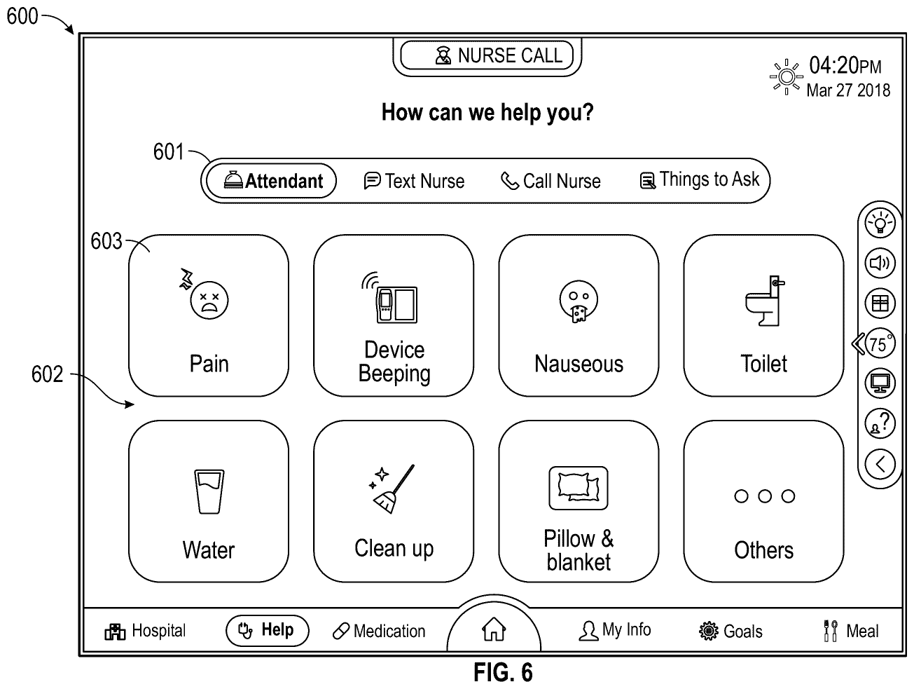

FIG. 6 shows a default Help tab screen 600, according to some configurations. The Help screen 600 may be accessed via the application navigation panel 205. The top control panel 601 may lead to sub-screens that allow patients to request staff assistance, contact a nurse, or write down questions to ask a care team member. The assistance buttons 602 may allow patients to specify the reason they need assistance, such that the appropriate hospital staff can be sent to the patient's room. In some configurations, when the patient selects an assistance button 602, a pop-up window or sub-screen may appear in which the patient can enter details about the selection. For example, after selecting the “nauseous” button, the physiological patient monitoring system may prompt the patient about the type of nausea, other discomfort associated with the nausea, etc. The physiological patient monitoring system may present pre-set options from which the patient may select, or the patient may freely type in details. Upon receiving user input, the physiological patient monitoring system may send an alert to one or more pagers, computing devices, portable electronic devices, or any other type of electronic device. The one or more electronic devices may be associated with a department in the hospital or with specific hospital staff. In some configurations, an individual electronic device may have a static pairing with one or more of the assistance buttons 602. In other configurations, the button-device pairings may be configurable, such that the individual electronic device may be used in different departments or by different hospital staff members. With the current predominant system, patients have one universal button that pages the nursing staff, even though most patient pages are non-emergencies, such as bedding adjustments, room operation questions, food requests, or cleaning requests. The assistance buttons 602 can save nurses and physicians time by sending the appropriate hospital staff from the start.

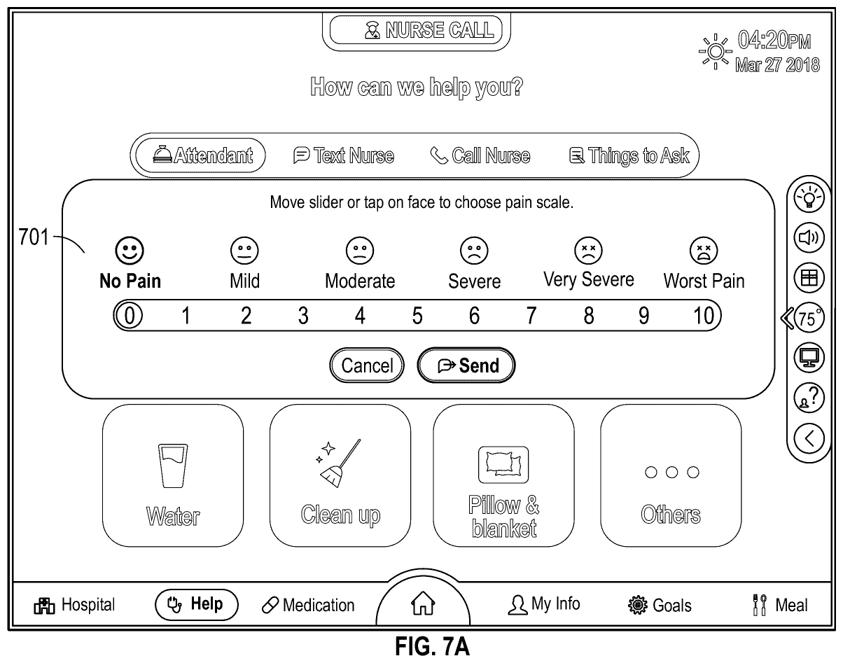

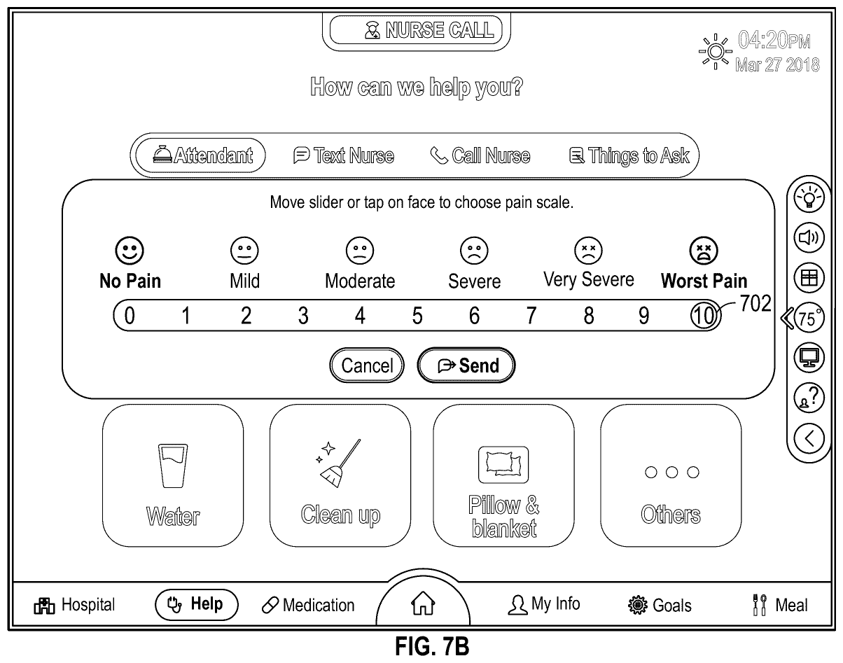

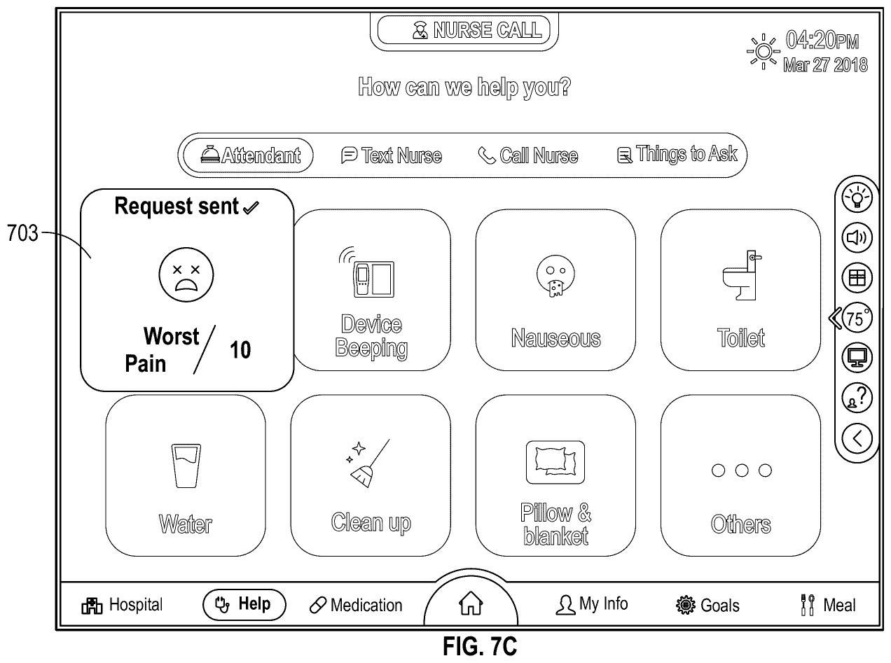

FIGS. 7A-7C illustrate how the Pain button 603 in FIG. 6 may work. FIG. 7A shows the pop-up 701 that may appear when the patient requests attendance due to pain. The pop-up 701 may be a numerical scale that reflects the severity of a patient's experienced pain. The numerical scale may span any number range and may be marked with any regular increment. In some configurations, the numerical scale may be controlled by a slider on a sliding scale rather than discrete buttons on an incremented scale. In some configurations, the pop-up may only display figures that reflect degrees of discomfort, may only contain descriptor words that reflect degrees of discomfort, or may be any other scale that allows patients to communicate their discomfort level. FIG. 7B illustrates how the graphics may update to reflect a patient selection 702. In some configurations, the pop-up may offer further patient prompts, such as location of pain, type of pain, duration of pain, etc. The physiological patient monitoring system may present pre-set options from which the patient may select, or the patient may freely type in details. In some configurations, the pop-up 701 may be a separate sub-screen. FIG. 7C shows an example of an updated Pain button 703 after the patient selection 702 is sent. The updated Pain button 703 may show the patient's selection and confirm the information was submitted.

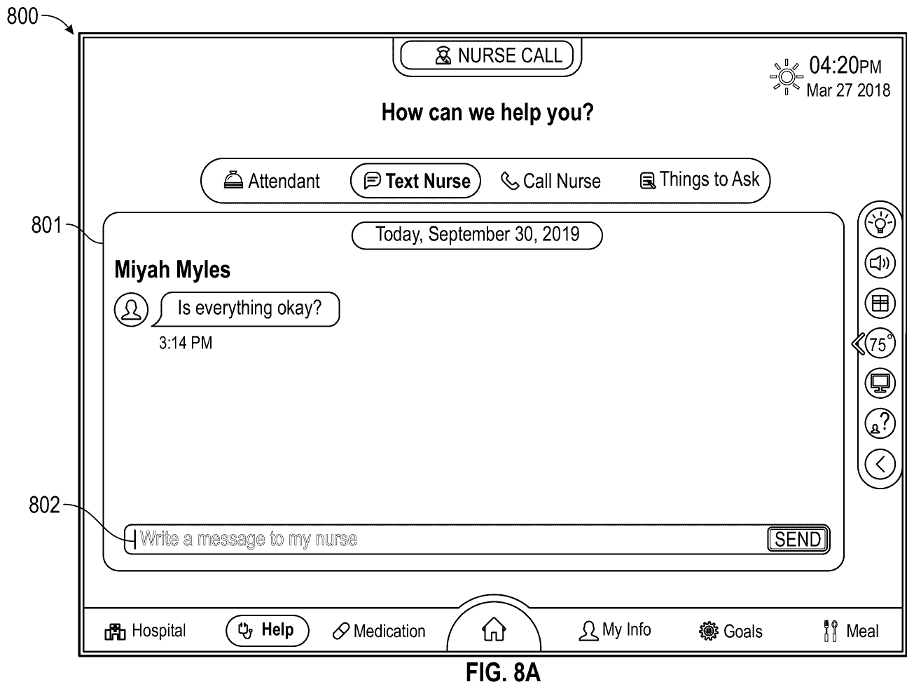

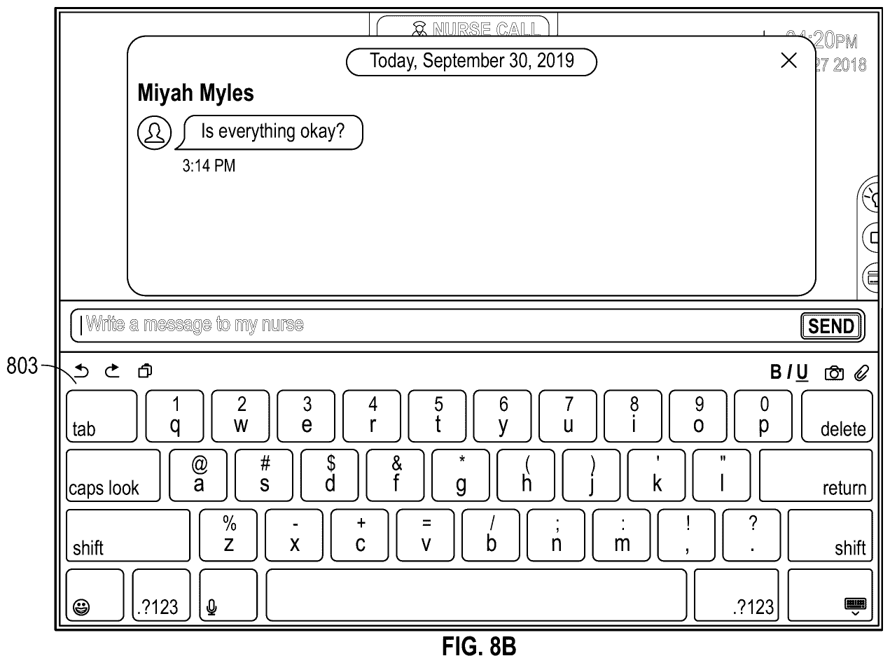

FIGS. 8A-8B illustrate an example Text Nurse sub-screen 800, which may be accessed through the control panel 601. FIG. 8A shows an illustrative chat window 801 which may open when the patient chooses Text Nurse. The chat function may be linked to an application on the nurse's cellular phone or other electronic device so that the nurse can receive live notifications and can provide live feedback. The Text Nurse feature may be linked to a device only accessible by the patient's attending nurse or may be linked to a general nursing department device such that any nurse may respond to the patient. FIG. 8B demonstrates an example virtual keyboard 803 that may appear when the patient taps on a message drafting space 802 on the chat window 801.

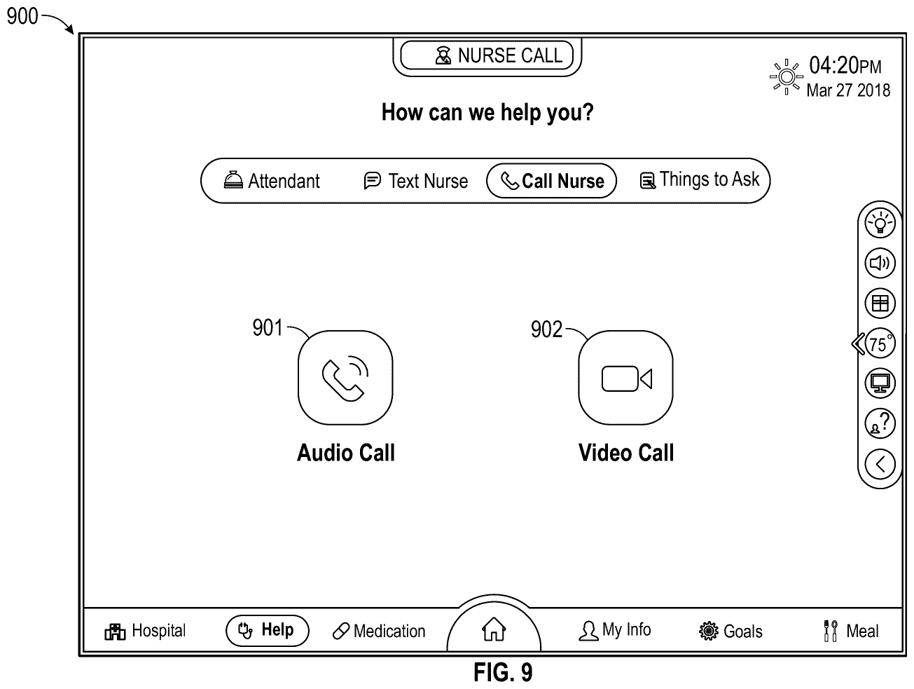

FIG. 9 illustrates an example Call Nurse sub-screen 900, which may be accessed through the control panel 601. The sub-screen may offer the options to either audio call 901 or video call 902 the nurse. The portable device 100 may thus have a microphone, a camera, or both such that the patient can call the nurse. If the portable device 100 does not support audio or video transmission, the call buttons may be disactivated or a warning pop-up may appear when the buttons are selected. Both call options may be linked to an application on the nurse's cellular phone or other electronic device so that the nurse can receive live notifications and can provide live feedback. The Call Nurse feature may be linked to a device only accessible by the patient's attending nurse or may be linked to a general nursing department device such that any nurse may respond to the patient.

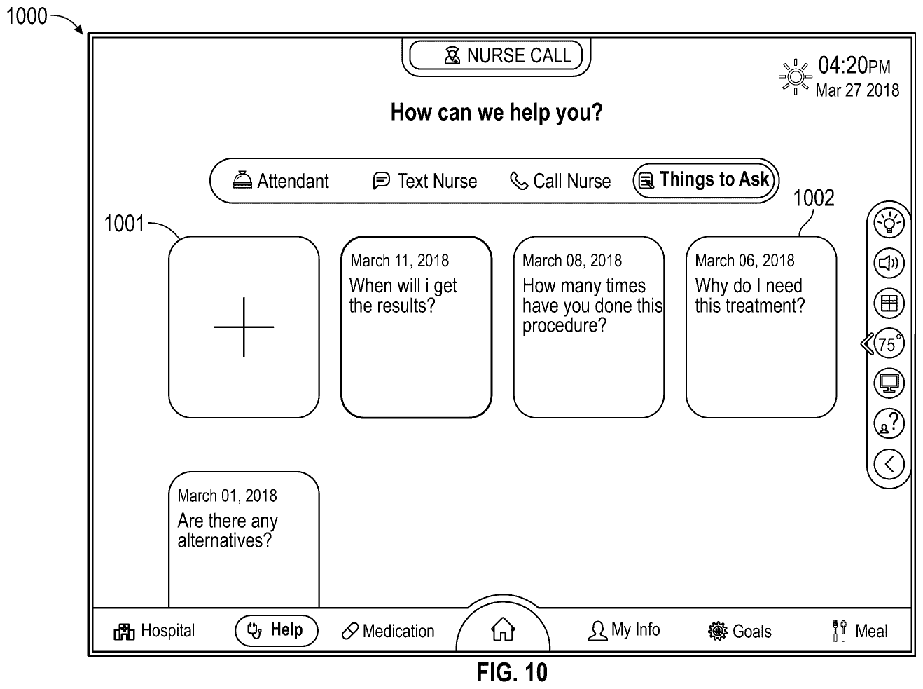

FIG. 10 illustrates an example Things To Ask sub-screen 1000, which may be accessed through the control panel 601. The Things To Ask sub-screen 1000 can function like a digital notepad where the patient can create new questions 1001 and view saved questions 1002. In some configurations, patients may be able to select a specific individual on their care team to direct each question. Saving questions until the staff's scheduled visit may be more efficient than paging staff members each time the patient thinks of a question. In some configurations, the patient interface of the physiological patient monitoring system may be linked to a healthcare provider interface such that healthcare providers may directly access the patient's questions from a separate electronic device.

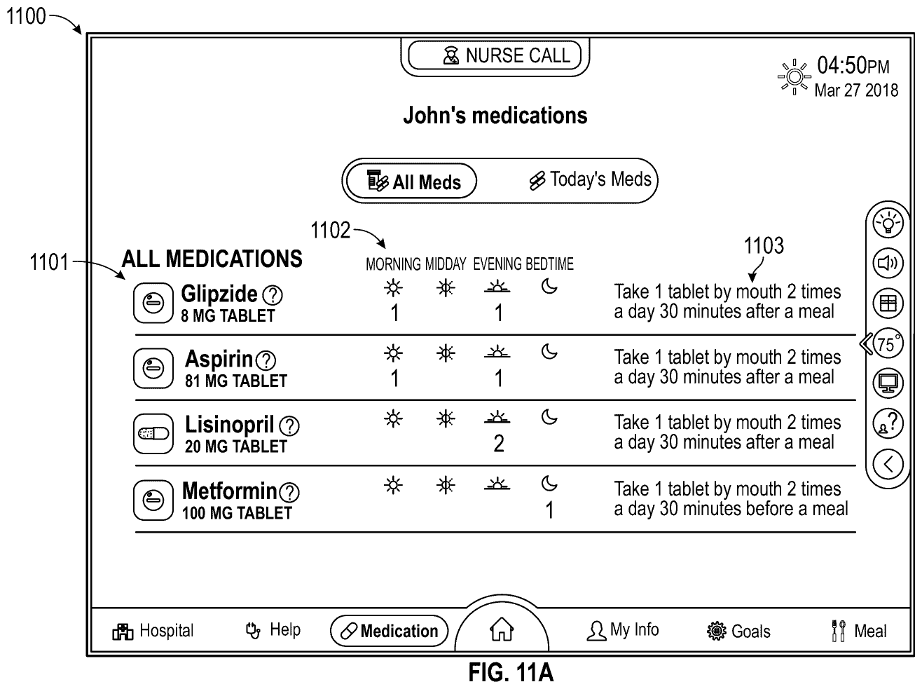

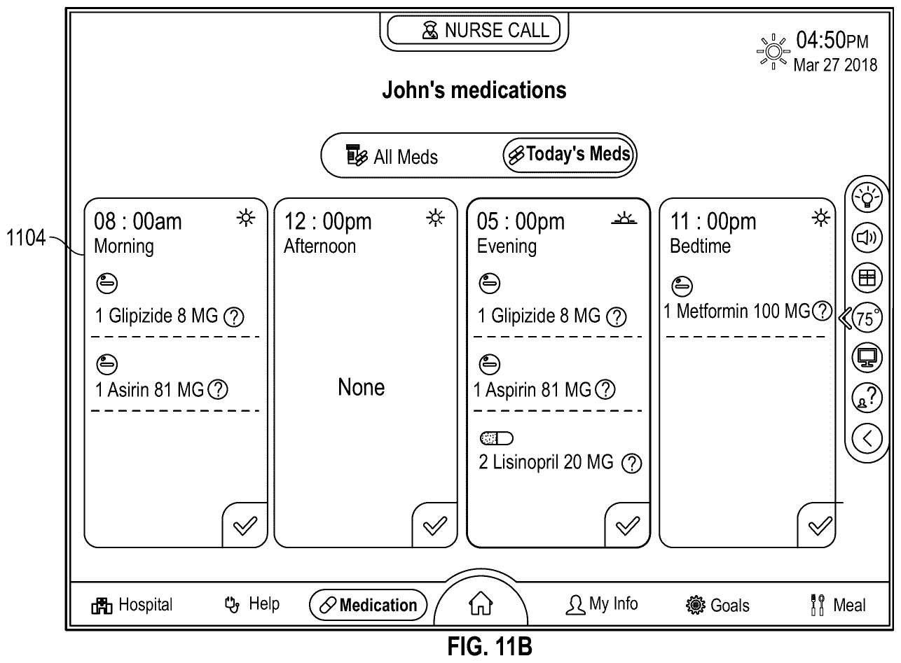

FIGS. 11A-11B show illustrative Medication tab sub-screens. FIG. 11A illustrates an example Medication tab default screen 1100. The Medication tab default screen 1100 may be accessed via the application navigation panel 205. The default screen 1100 may list all the patient's medications and related details, such as drug name 1101, administration schedule 1102, and dosage instructions 1103. The patient may also tap on individual drugs on the drug name list to view extra drug information, such as what the drug may treat, side effects of the drugs, and other information typically included on drug labels. In some configurations, an icon may be included that accurately reflects the physical form or appearance of the drug. FIG. 11B may offer a detailed view 1104 of the medication administration schedule 1102 for a given day, broken down by time and dosage. In some configurations, the detailed view 1104 displays medication information for the current day. In other configurations, the detailed view may include a date selection feature where patients can view their medication details for other dates. The detailed view 1104 may also include icons that show the physical form of the drugs. The information presented in the Medication tab sub-screens can not only help patients better understand what medications they are taking but can also act as a simple method for healthcare providers to confirm they are administering the correct medications.

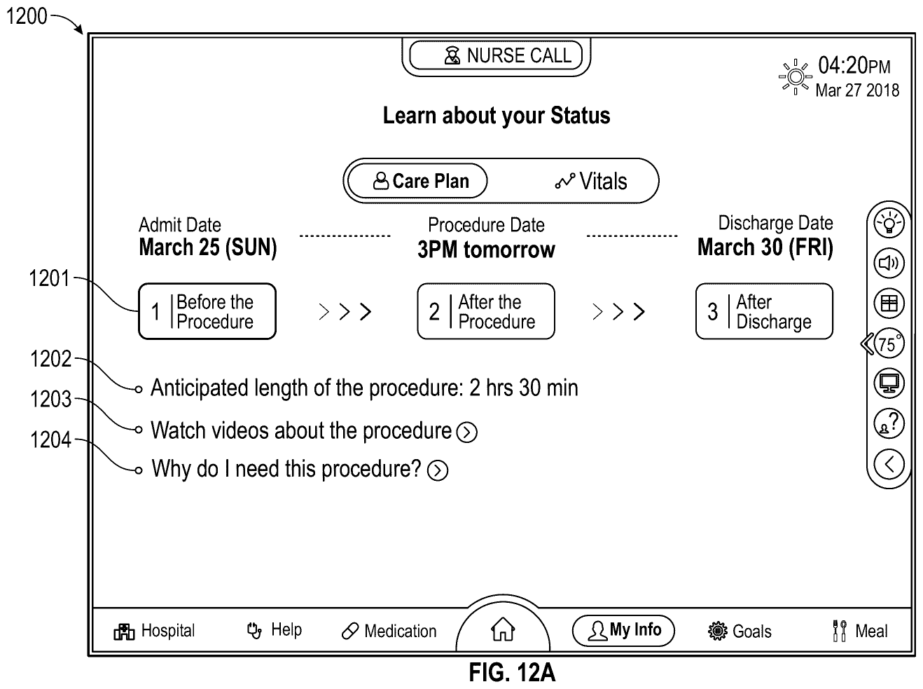

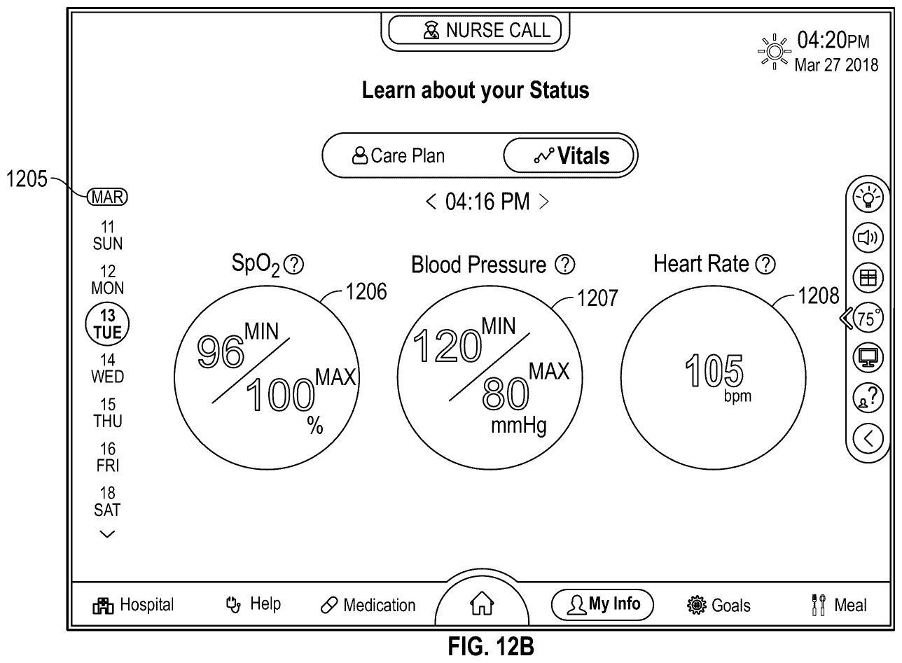

FIGS. 12A-12B show an illustrative My Info tab sub-screens. FIG. 12A illustrates an exemplary My Info tab default screen 1200, or Care Plan sub-screen, which may outline the patient's care plan. The My Info tab default screen 1200 may be accessed via the application navigation panel 205. The Care Plan sub-screen may show a procedure timeline 1201 and procedure details, including the length of the procedure 1202, videos about the procedure 1203, and why the procedure is necessary in the patient's case 1204. The procedure timeline 1201 may have buttons associated with each event on the timeline, and the patient may tap on each button to access an interactive sub-screen to learn more about each individual event. For example, a Before the Procedure sub-screen may allow the patient to access pre-approved informational videos by tapping on the button for videos about the procedure 1203. This provides approved information to patients, avoiding misinformation through a patient's own independent research, which can potentially carry the risk of finding the wrong procedure or being misinformed. Similar content may be incorporated in the other procedure timeline sub-screens. The content on the Care Plan sub-screen 1200 may also be customized for the patient. For example, a feature explaining why the procedure is necessary in the patient's case 1204 could be a short explanation personally drafted by the attending physician. Personalized explanations can help patients feels more at ease and more informed. FIG. 12B shows an example Vitals sub-screen, which may track the patient's oxygen levels 1206, blood pressure 1207, and heart rate 1208. The vitals may be recorded, so the patient can use the calendar panel 1205 to view trends.

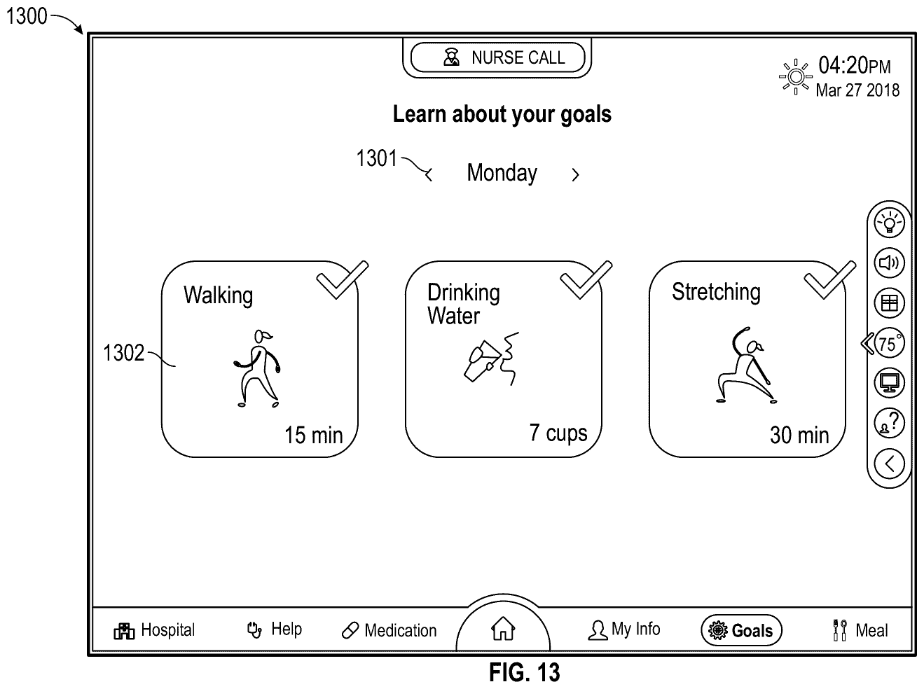

FIG. 13 illustrates an example default Goals tab screen 1300. Patients can check their daily tasks and completion statuses for those tasks. The Goals screen 1300 may be accessed via the application navigation panel 205. A date selector 1301 may allow patients to review old goals and see upcoming goals. The date selector 1301 may be a scroll-through list, a drop-down menu, a calendar graphic with selectable dates, or any other graphical representation that can allow the patient to view data from any date during the patient's hospital stay. The goals display 1302 may update to mark tasks as completed. Tasks may be marked completed by either patients or healthcare providers, or both.

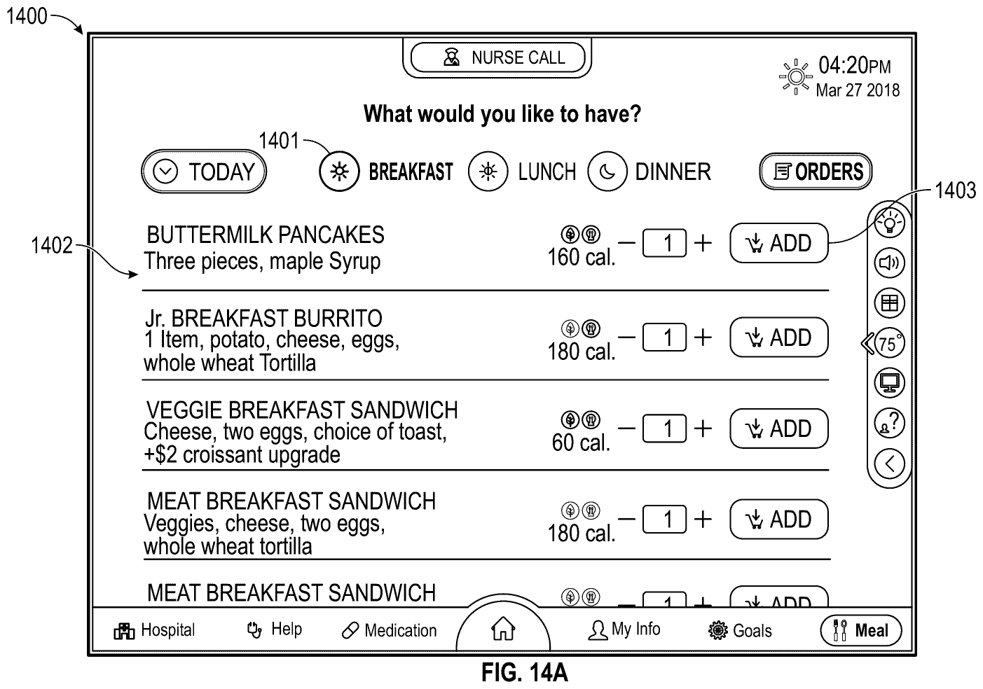

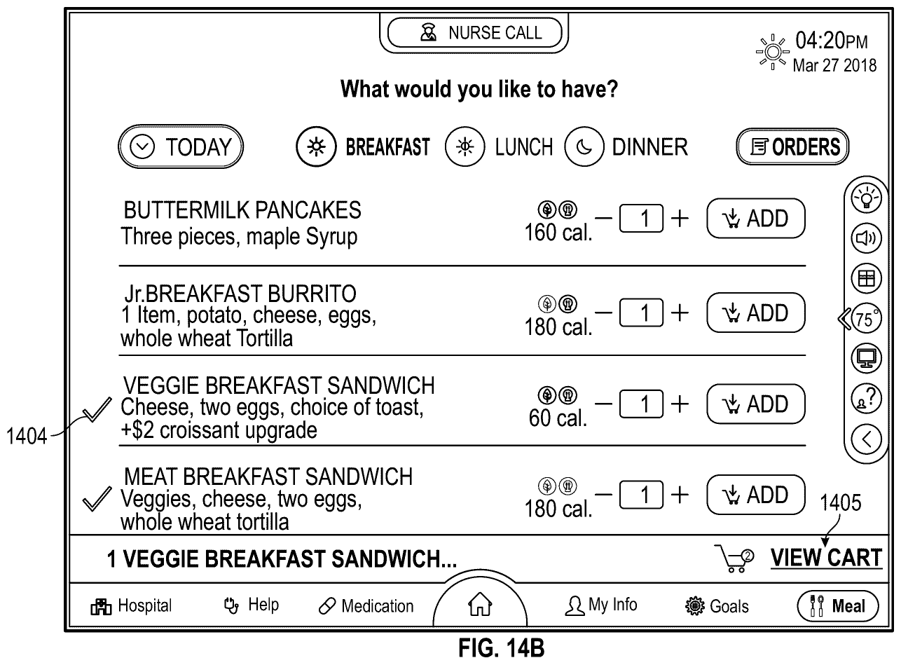

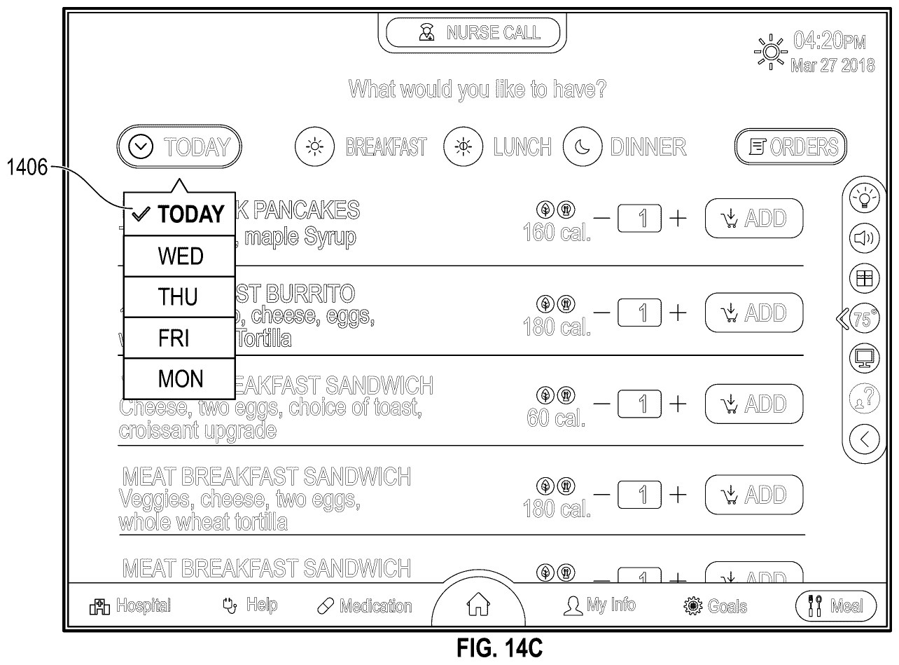

FIGS. 14A-14C show an exemplary Meal tab screen 1400 through which the patient can order food for any meal of the week. The Meal screen 1400 may be accessed via the application navigation panel 205. FIG. 14A shows an illustrative Meal tab screen 1400 before the patient places an order. The application may allow for meal selection 1401, so patients can order food for any meal of the day. The menu 1402 may list details about each dish, including, but not limited to, caloric and allergen information. When ready, the patient can tap the add button 1403 to add the dish to a virtual cart 1405. FIG. 14B shows an illustrative Meal tab screen 1400 after the patient has selected menu items. The selection graphic 1404 may update to show which dishes have been selected, and the patient can see the full list in a virtual cart 1405. FIG. 14C may show a date selection menu 1406 that the patient could use to order food for different days of the week. The date selection menu 1406 may be a scroll-through list, a drop-down menu, a calendar graphic with selectable dates, or any other graphical representation.

The various features and processes described above may be used independently of one another or may be combined in various ways. All possible combinations and sub-combinations are intended to fall within the scope of this disclosure. In addition, certain interfaces and features may be omitted in some implementations. The interfaces described herein are also not limited to any particular sequence and may be arranged in other sequences that are appropriate. Features may be added to or removed from the disclosed example configurations. The example systems and components described herein may be configured differently than described. For example, elements may be added to, removed from, or rearranged compared to the disclosed example configurations

Conditional language, such as, among others, “can,” “could,” “might,” or “may,” unless specifically stated otherwise, or otherwise understood within the context as used, is generally intended to convey that certain configurations include, while other configurations do not include, certain features, elements and/or steps. Thus, such conditional language is not generally intended to imply that features, elements and/or steps are in any way required for one or more configurations or that one or more configurations necessarily include logic for deciding, with or without user input or prompting, whether these features, elements and/or steps are included or are to be performed in any particular configuration.

It should be emphasized that many variations and modifications may be made to the above-described configurations, the elements of which are to be understood as being among other acceptable examples. All such modifications and variations are intended to be included herein within the scope of this disclosure. The foregoing description details certain configurations of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

1. A hospital patient assistance system comprising:
a portable electronic device;
said portable electronic device comprising a display and one or more hardware processors configured to:
generate one or more graphical user interfaces for presentation on the display, said one or more graphical user interfaces configured to display a plurality of patient assistance options, wherein the patient assistance options includes an emergency nurse call feature and a non-emergency nurse call feature.

2. (canceled)

3. The system of claim 1, wherein the one or more hardware processors are further configured to:
receive, via the one or more graphical user interfaces, a user selection of a second patient assistance option;
generate a patient assistance request, wherein the patient assistance request comprises the user selection;
determine a recipient of the patient assistance request from a plurality of possible recipients based at least in part on the user selection; and
deliver the patient assistance request to a second electronic device, wherein the second electronic device is associated with the recipient.

4. The system of claim 1, wherein the one or more computer hardware processors are further configured to:
receive data from a healthcare data network, wherein the data comprises healthcare and treatment regimen information;
display the data from the healthcare data network in real time;
track user progress throughout a treatment regimen; and
update a representation of a living entity throughout the treatment regimen.

5. The system of claim 4, wherein the healthcare and treatment regimen information includes at least one of: treatment information personalized to the user, or general educational materials.

6. The system of claim 1, wherein the one or more computer processors are further configured to, in response to user input, interact with nearby electronically-controlled items.

7. The system of claim 4, wherein the living entity is a plant.

8. The system of claim 7, wherein living entity includes one or more leaves.

9. The system of claim 1, wherein the one or more user interfaces comprises icons selectable by the patient to indicating frequently used status indicators.

10. The system of claim 1, wherein a hospital room does not require physical switches for calling nurse.

11. The system of claim 1, wherein the patient assistance options further comprise a levels of pain feature allowing a user to indicate a level of pain which is communicated to a healthcare provider.

12. The system of claim 11, wherein the levels of pain feature communicates a level of pain to the healthcare provider when one or both of the emergency and non-emergency nurse call features are activated.
